(19) United States
(12) Patent Application Publication
(10) Pub. No.: US 2017/0266164 A1
(43) Pub. Date: Sep. 21, 2017

(54) NEW METHODS AND USES

(71) Applicant: Vicore Pharma AB, Molndal (SE)

(72) Inventor: Punam Malik, Cincinnati, OH (US)

(21) Appl. No.: 15/326,849

(22) PCT Filed: Dec. 11, 2015
(86) PCT No.: PCT/GB2015/053896

§ 371 Date: Jan. 17, 2017

(30) Foreign Application Priority Data
Dec. 12, 2014 (GB) 1422111.3

(51) Int. Cl. A61K 31/4178; A61K 45/06

(52) U.S. Cl. CPC A61K 31/4178; A61K 45/06

(57) ABSTRACT

This invention relates to a new use of compounds that are angiotensin II receptor agonists, specifically agonists of the angiotensin II type 2 receptor (the AT2 receptor), and especially agonists that bind selectively to the AT2 receptor, in the treatment of sickle cell disease.

This invention was made with government support under HL117709 awarded by the National Institutes of Health. The government has certain rights in the invention.

### FIELD OF THE INVENTION

The present invention relates to a new use of compounds that are angiotensin II receptor agonists, more particularly selective agonists of the angiotensin II type 2 receptor (hereinafter the AT2 receptor), and especially agonists that bind selectively to that receptor, for the treatment of subjects with sickle cell disease (SCD).

### BACKGROUND OF THE INVENTION

The listing or discussion of an apparently prior-published document in this specification should not necessarily be taken as an acknowledgement that the document is part of the state of the art or is common general knowledge

Sickle cell disease is a serious inherited blood disorder where the red blood cells, which carry oxygen around the body, develop abnormally. The disorder mainly affects people of African, Caribbean, Middle Eastern, Eastern Mediterranean and Asian origin. As used hereinafter the term “sickle cell disease” also may refer to homozygous sickle cell disease (hemoglobin [Hb] SS disease) and Hb S-β° thalassemia (Hb SS and Hb β30 thalassemia are grouped together as sickle cell anemia) or hemoglobin SC, SD and SE disease or other variants. The term sickle cell disease encompasses sickle cell anemia and all of these variants.

Normal red blood cells are flexible and disc-shaped, but in sickle cell disease they can become rigid and shaped like a crescent (or sickle). The sickle-shaped cells contain defective hemoglobin, the iron-rich protein that enables red blood cells to carry oxygen from your lungs to the rest of the body. The abnormal cells are also unable to move around as easily as normal shaped cells and can block blood vessels, resulting in tissue and organ damage and episodes of severe pain. Such episodes are known as a sickle cell crisis or a vaso-occlusive crisis. They can last from a few minutes to several months, although on average most last five to seven days.

The abnormal blood cells also have a shorter lifespan and are not replaced as quickly as normal blood cells. This leads to a shortage of red blood cells, known as anemia. Symptoms of anemia include lethargy (a lack of energy), tiredness and breathlessness, particularly after exercise.

The life expectancy of subjects with sickle cell disease has improved considerably since its identification in 1960. In the developed countries, such as the United States, the life expectancy of patients with sickle cell disease is 45-48 years as compared to 78 years of African Americans without sickle cell disease. Recent studies have shown that approximately 85 percent of children and adolescents with sickle cell anemia (homozygous for sickle hemoglobin) and 95 percent of subjects with sickle cell-hemoglobin C disease (heterozygous for hemoglobin S and C) survive to 20 years of age. However, it remains the case that the disease dramatically reduces the life expectancy of the sufferer. In the developing countries such as Africa, most patients with sickle cell disease seldom survive beyond the age of 5-10 years.

Treatment strategies for sickle cell disease vary from the use of drugs to transfusion therapy or bone marrow transplants, a bone marrow transfusion providing the only known cure. Hydroxyurea, a drug that increases fetal hemoglobin production, ameliorates disease symptoms, if taken daily for life.

With improved medical care, largely preventing infection related deaths in sickle cell disease, patients are surviving to adulthood. This is when the chronic end organ damage is becoming apparent. Nearly 30-50% of adults with sickle cell disease develop nephropathy and eventual end stage renal disease, a common cause of death in adults with sickle cell disease.

The inventors have recently discovered that the renin-angiotensin system (RAS) is highly activated in sickle cell disease and this activation has a dual effect: Increased RAS signaling improves urine concentrating ability (UCA), but also results in glomerular damage—albuminuria and focal segmental glomerular sclerosis and nephron loss.

Renin, a protease, cleaves its only known substrate (angiotensinogen) to form angiotensin I, which in turn serves as substrate to angiotensin converting enzyme (ACE) to form Angiotensin II (Ang II). The endogenous hormone Ang II is a linear octapeptide (Asp¹-Arg²-Val³-Tyr⁴-Ile⁵-His⁶-Pro⁷-Phe⁸), and is an active component of the renin angiotensin system (RAS). The AT1 receptor is expressed in most organs, and is believed to be responsible for the majority of the pathological effects of Ang II.

Several studies in adult individuals appear to demonstrate that, in the modulation of the response following Ang II receptor stimulation, activation of the AT2 receptor has opposing effects to those mediated by the AT1 receptor. The AT2 receptor has also been shown to be involved in apoptosis and inhibition of cell proliferation (de Gasparo M et al. Pharmacol Rev 2000; 52:415-472).

More recently, AT2 receptor agonists have been shown to be of potential utility in the treatment and/or prophylaxis of disorders of the alimentary tract, such as dyspepsia and irritable bowel syndrome, as well as multiple organ failure (see international patent application WO 99/43339). The expected pharmacological effects of agonism of the AT2 receptor are described in general in de Gasparo M et al., 2000. It is not mentioned that agonism of the AT2 receptor may be used to treat SCD.

The effects of Ang II on cell growth, inflammation and extracellular matrix synthesis are mainly coupled to AT1, whereas the function of AT2 has been heavily investigated and new research indicates that it is more prevalent in damaged tissue and exerts reparative properties and properties opposing the AT1 receptor. The AT2 receptor has been shown to be of importance in relation to reduction of myocyte hypertrophy and fibrosis.

AT2 receptor agonists have also been described in the prior art, for instance in international patent application WO 2002/096883.

Stimulation of the AT2 receptor with C21 (as defined herein) ameliorates LV fibrosis by regulation of tissue inhibitor of Matrix Metalloproteinase 1/Matrix Metalloproteinase 9 and TGF (transforming growth factor) β₁ in rat heart (Lauer et al. Hypertension 2014, 63: 60-67) and has also been demonstrated in the treatment of cerebral malaria (WO 2013/158628).

The effects of angiotensin-(1-7) receptor agonists (compounds that have a positive impact on the function of an angiotensin-(1-7) receptor) on graft versus host disease is described in WO2013/158959.

### SUMMARY OF THE INVENTION

Compounds of the invention are angiotensin II receptor agonists, more particularly, are agonists of the AT2 receptor, and, especially, are selective agonists of that sub-receptor. In some embodiments, the compounds of the invention are those that can selectively stimulate AT2 receptors.

In one aspect of the present invention, there is provided a method of treatment of SCD, which method comprises administration of a therapeutically effective amount of a compound of the invention (or a pharmaceutically acceptable salt, solvate or prodrug thereof) to a subject suffering from SCD.

In some embodiments, the compound can be N-butyloxycarbonyl-3-(4-imidazol-1-ylmethylphenyl)-5-iso-butylthiophene-2-sulfonamide (also referred to as C21) or a pharmaceutically acceptable salt, solvate or prodrug thereof.

Other embodiments and advantages will be more fully apparent from the following disclosure.

### BRIEF DESCRIPTION OF THE DRAWINGS

The following drawings are provided to illustrate various aspects of the present inventive concept and are not intended to limit the scope of the present invention unless specified herein.

FIG. 1 presents the structure of Compound 21, or in short C21, which is N-butyloxycarbonyl-3-(4-imidazol-1-ylmethylphenyl)-5-iso-butylthiophene-2-sulfonamide.

### DETAILED DESCRIPTION OF THE INVENTION

The foregoing and other aspects of the present invention will now be described in more detail with respect to the description and methodologies provided herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

The inventors have found that while they prevent development of sickle nephropathy with AT1 receptor blockade using pharmacological agents (ACE inhibitors or AT1R blockers) or genetic approaches (using mouse models of sickle cell disease in an AT1R knockout background) in mouse models of sickle cell disease, this results in worsening of urine concentration ability. Using mice with germline AT1 or AT2 receptor deficiencies with sickle cell disease, they discovered the role of AT2 receptor in urine concentration has not been previously identified. The inventors were able to improve the urine concentrating ability of sickle mice with an AT2 receptor agonist, C21.

The inventors have surprisingly found that compounds that are angiotensin II agonists, and more particularly selective agonists of the angiotensin II type 2 receptor (hereinafter the AT2 receptor), and especially agonists that bind selectively to that receptor, are of use in the treatment of sickle cell disease (SCD).

As used herein, compounds that are angiotensin II receptor agonists may be referred to as “compounds of the invention”.

Thus, in a first aspect of the invention, there is provided a method of treatment of SCD, which method comprises administration of a therapeutically effective amount of an angiotensin II receptor agonist, or a pharmaceutically acceptable salt, solvate or prodrug thereof, to a subject suffering from SCD.

In an alternative first aspect of the invention, there is provided an angiotensin II receptor agonist, or a pharmaceutically acceptable salt, solvate or prodrug thereof, for use in the treatment of SCD.

In a further alternative first aspect of the invention, there is provided the use of an angiotensin II receptor agonist, or a pharmaceutically acceptable salt, solvate or prodrug thereof, in the manufacture of a medicament for the treatment of SCD.

Subjects suffering from SCD may have impaired urine concentrating ability, and the inventors have found that urine concentrating ability may be improved using the treatments described here, thereby treating a symptom effect of SCD. Thus in an embodiment of the first aspects of the invention, the treatment of sickle cell disease results in an improvement in the urine concentrating ability in a subject.

Furthermore, while each of the aspects of the invention as described herein relates to the treatment of sickle cell disease, the invention also relates to corresponding methods, compounds for use, formulations for use, combination products for use, and uses which relate to improving the urine concentrating ability in a subject. Thus, in an alternative aspect of the invention there is provided a method of improving the urine concentrating ability in a subject which method comprises administration of a therapeutically effective amount of an angiotensin II receptor agonist, or a pharmaceutically acceptable salt, solvate or prodrug thereof, to a subject suffering from SCD.

The skilled person will understand that terminology used in the description of the invention herein is for the purpose of describing particular embodiments only and is not intended to be limiting of the invention. Unless otherwise defined, all terms, including technical and scientific terms used in the description, have the same meaning as commonly understood by one of ordinary skill in the art to which this invention belongs.

As used in the description of the embodiments of the invention, the singular forms “a” “an” and “the” are intended to include the plural forms as well, unless the context clearly indicates otherwise. Thus, such references may be replaced with a reference to “one or more” (e.g. one) of the relevant component or integer.

As used herein, all references to “one or more” of a particular component or integer will be understood to refer to from one to a plurality (e.g. two, three or four) of such components or integers. It will be understood that references to “one or more” of a particular component or integer will include a particular reference to one such integer.

Also, as used herein, “and/or” refers to and encompasses any and all possible combinations of one or more of the associated listed items. Furthermore, the term “about,” as used herein when referring to a measurable value such as an amount of a compound, dose, time, temperature, and the like, refers to variations of 20%, 10%, 5%, 1%, 0.5%, or even 0.1% of the specified amount.

When a range is employed (e.g. a range from x to y) it is it meant that the measurable value is a range from about x to about y, or any range therein, such as about x₁ to about y₁, etc.

It will be further understood that the terms “comprises” and/or “comprising,” when used in this specification, specify the presence of stated features, integers, steps, operations, elements, components and/or groups thereof, but do not preclude the presence or addition of one or more other features, integers, steps, operations, elements, components, and/or groups thereof.

By the term “treat,” “treating,” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

The term “therapeutically effective” as used herein in reference to an amount or dose refers to an amount of a compound, composition and/or formulation of the invention that is sufficient to produce a desired effect, which can be a therapeutic and/or beneficial effect. Such an effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount in any individual case can be determined by one skilled in the art by reference to the pertinent texts and literature and/or by using routine experimentation.

A “subject in need” of the methods of the invention can be a subject known to have or suspected of having SCD.

Subjects suitable to be treated with compounds and formulations of the present invention as described herein include, but are not limited to, mammalian subjects. In some embodiments, the subject may be a human subject.

As used herein, references to a “subject” to be treated may be synonymous with a “patient”, and vice versa.

As used herein the term “concomitant administration” or “combination administration” or the like of a compound, therapeutic agent or known drug with a compound of the present invention means administration of a known medication or drug to a subject and, in addition, the administration of one or more compounds of the invention to the same subject at such time that both the known drug and the compound will have a therapeutic effect. In some cases this therapeutic effect will be synergistic

Such concomitant administration can involve concurrent (i.e. at the same time), prior, or subsequent administration of the known drug with respect to the administration of a compound of the present invention. A person skilled in the art, would have no difficulty determining the appropriate timing, sequence and dosages of administration for particular drugs and compounds of the present invention.

Pharmaceutically-acceptable salts include, but are not limited to, acid addition salts and base addition salts. Such salts may be formed by conventional means, for example by reaction of a free acid or a free base form of a compound of the invention with one or more equivalents (as required) of an appropriate acid or base, optionally in a solvent, or in a medium in which the salt is insoluble, followed by removal of said solvent, or said medium, using standard techniques (e.g. in vacuo or by freeze-drying). Salts may also be prepared by exchanging a counter-ion of a compound of the invention in the form of a salt with another counter-ion, for example using a suitable ion exchange resin. For the avoidance of doubt, other pharmaceutically acceptable derivatives of compounds of the invention are included within the scope of the invention (e.g. solvates, prodrugs etc).

In particular embodiments, the pharmaceutically-acceptable salt is an HCl salt (i.e. an HCl salt of the compound of the invention).

As used herein, a “prodrug” is a composition that undergoes an in vivo modification when administered to a subject, wherein the product of the in vivo modification is a therapeutically effective compound. Prodrugs of compounds may be prepared by, for example, preparing a given compound as an ester. Thus, for example, an esterified form of the compound may be administered to a subject and may be de-esterified in vivo thereby releasing a therapeutically effective compound. Alternatively, some compounds may be prepared as prodrugs by adding short polypeptides (e.g. 1-6 amino acids) to the compound. Such prodrugs when administered to a subject may be cleaved (by, for example, trypsin or other peptidases/proteases) thereby releasing a therapeutically effective compound. Formation of prodrugs is not limited by the specific examples described herein. Other ways of preparing therapeutically effective compounds as prodrugs are known.

Compounds of the invention may exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

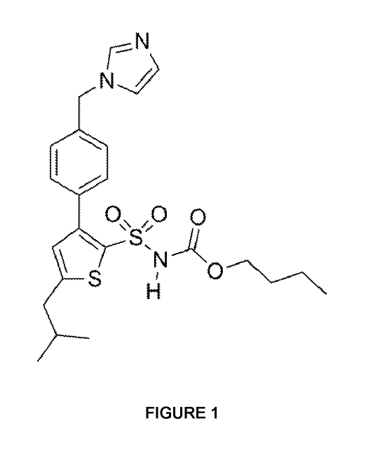

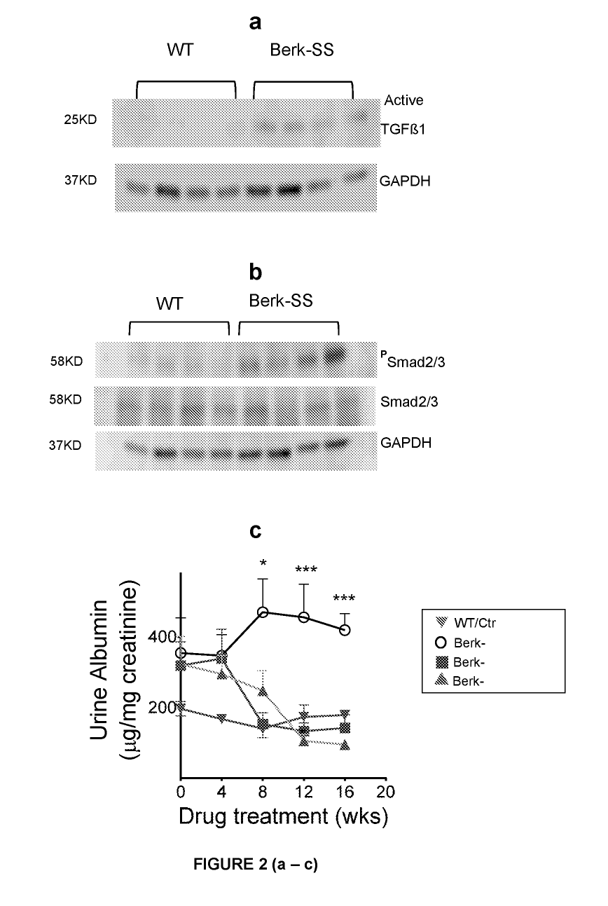

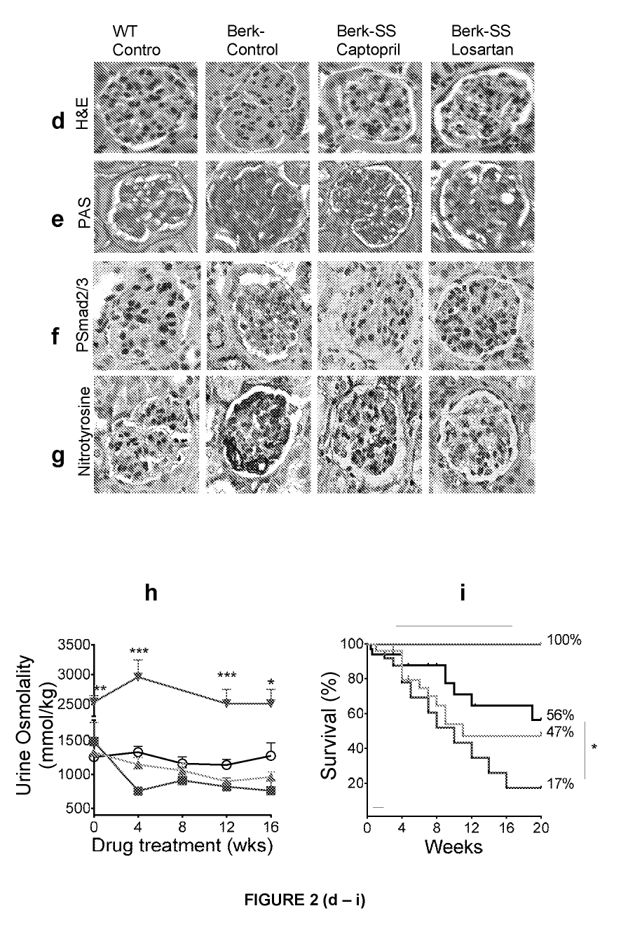

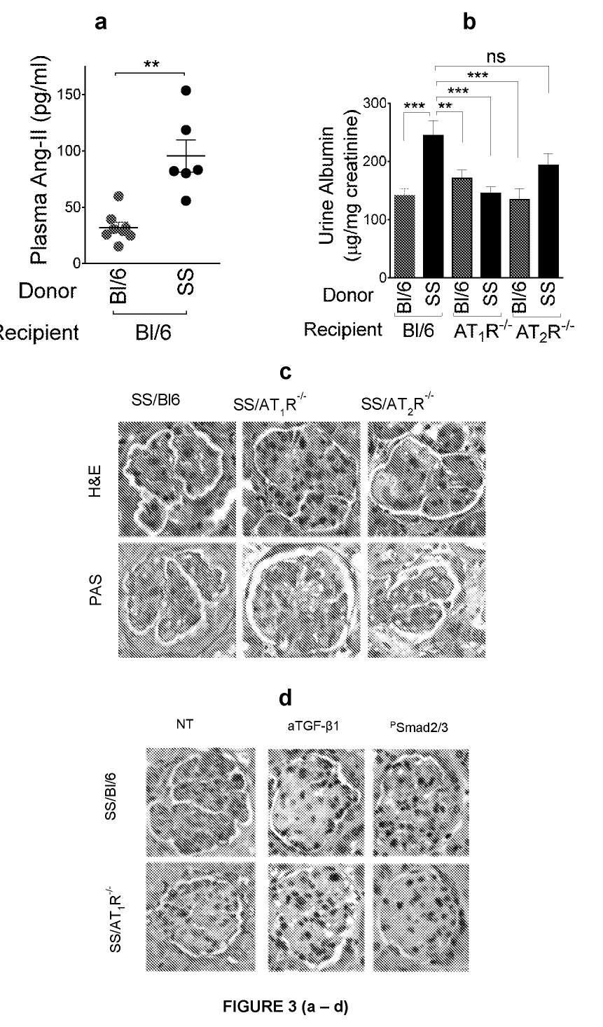

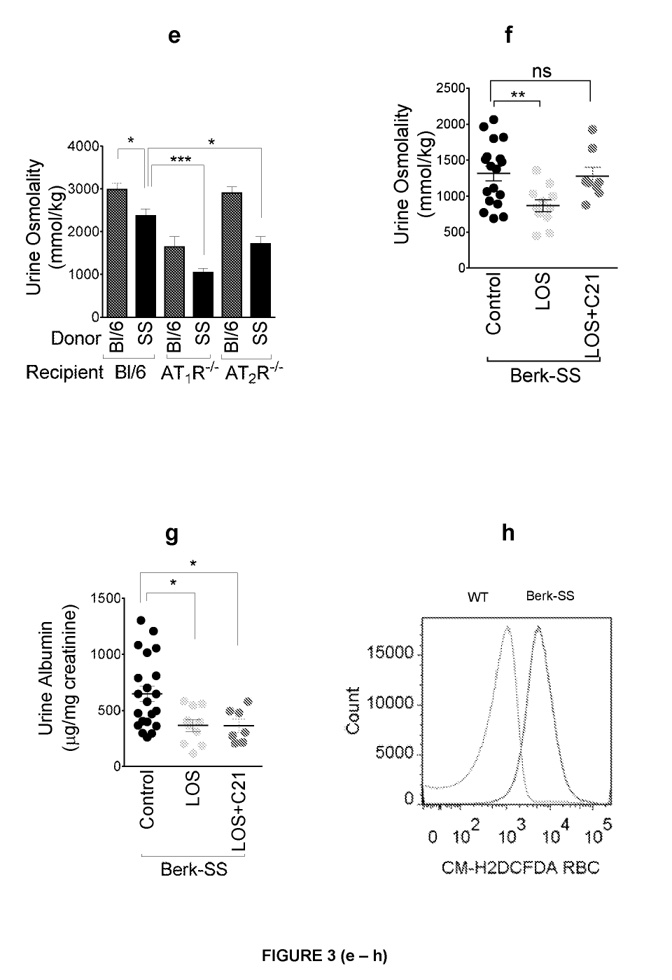

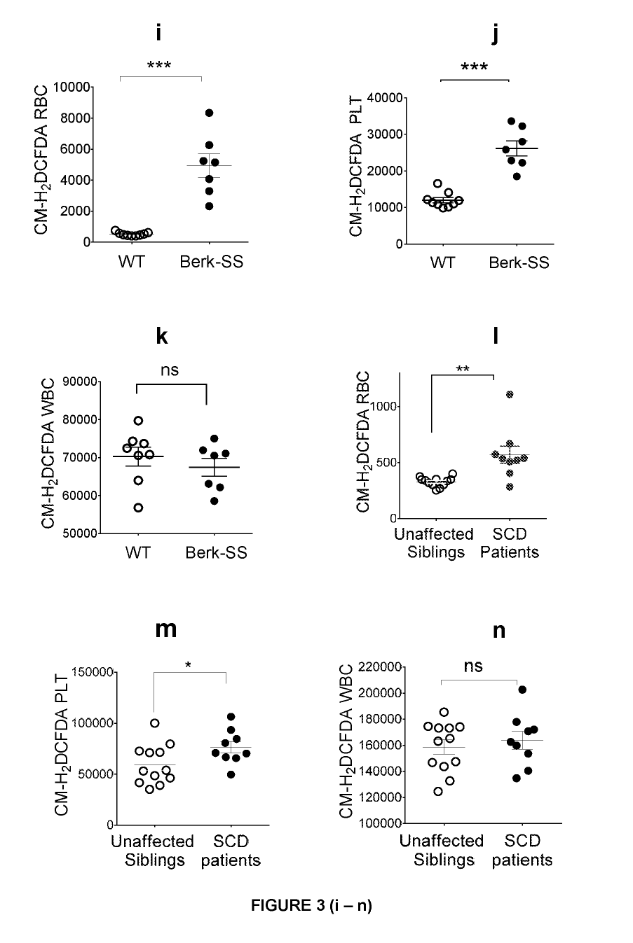

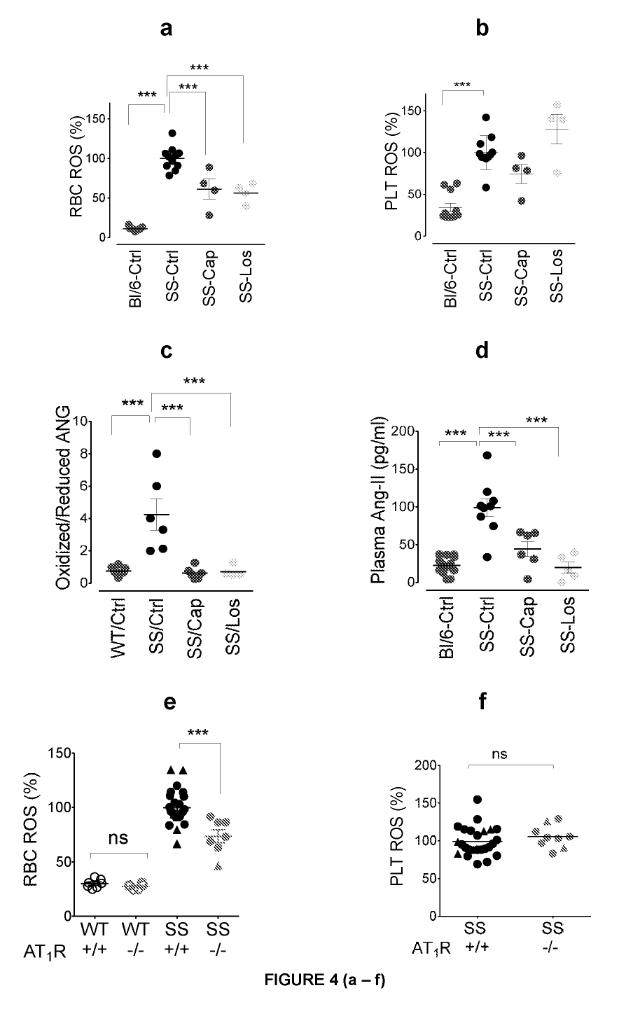

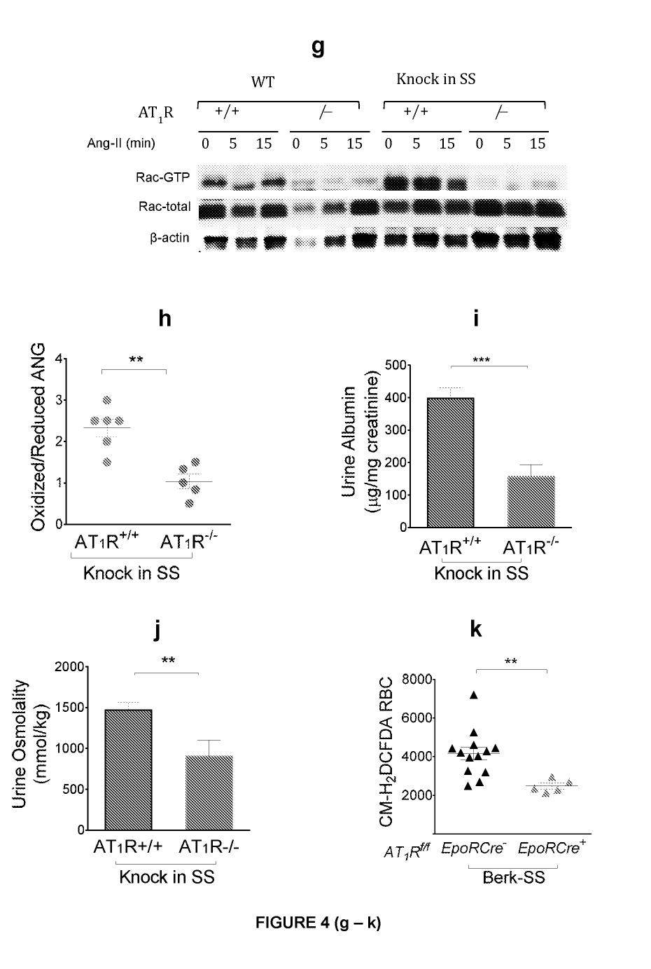

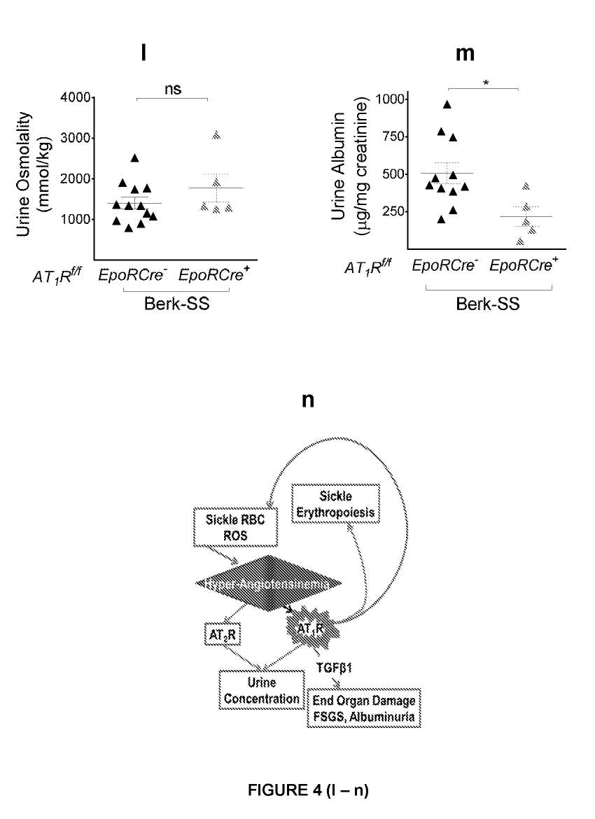

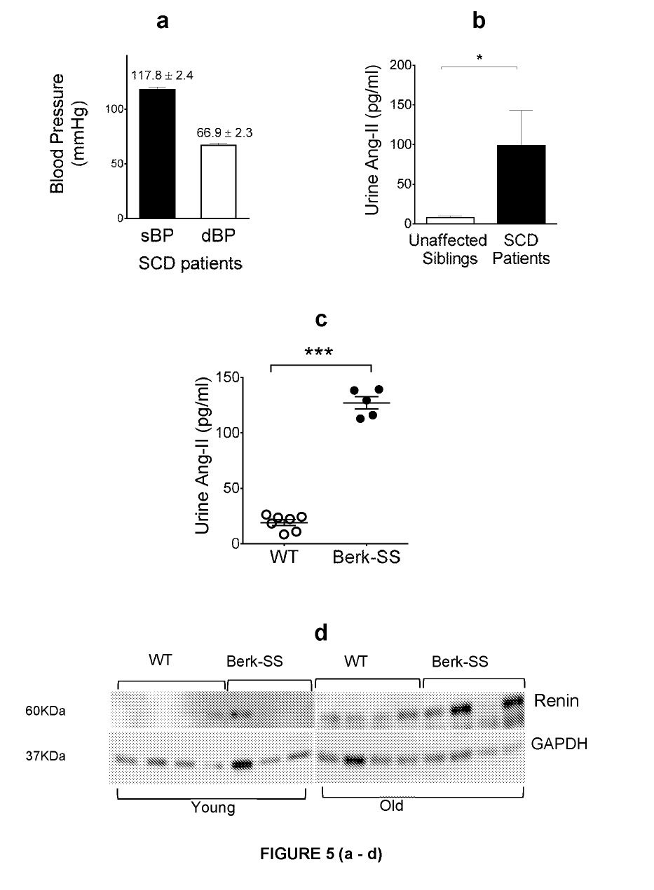

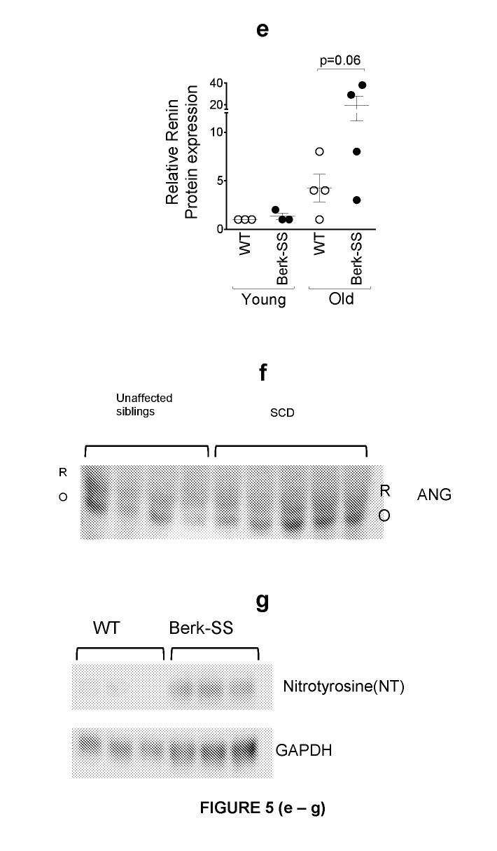

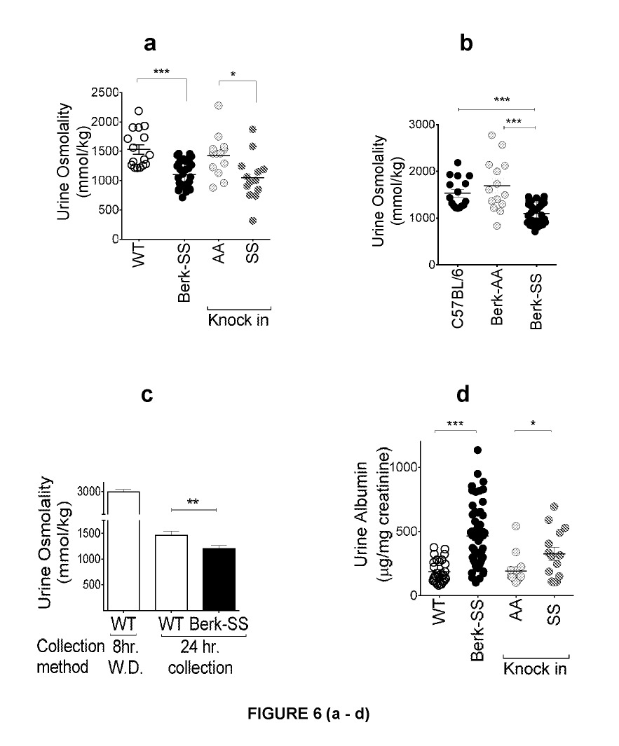

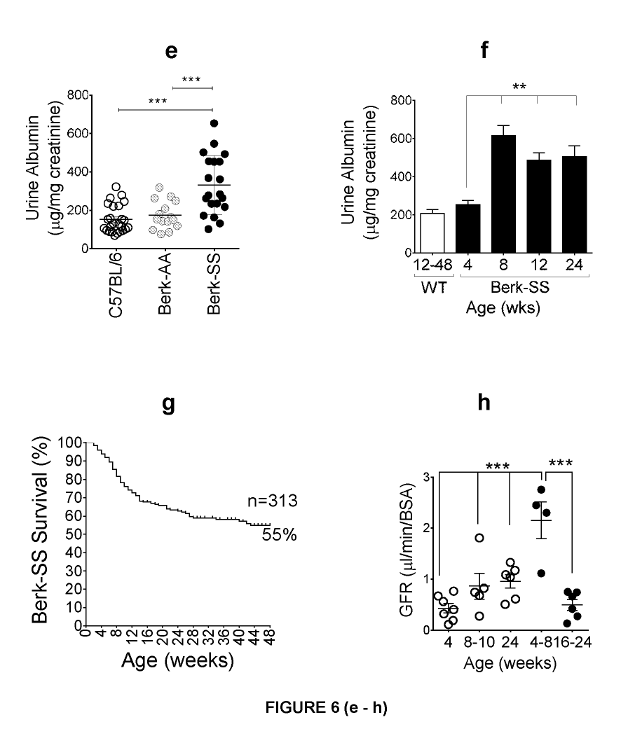

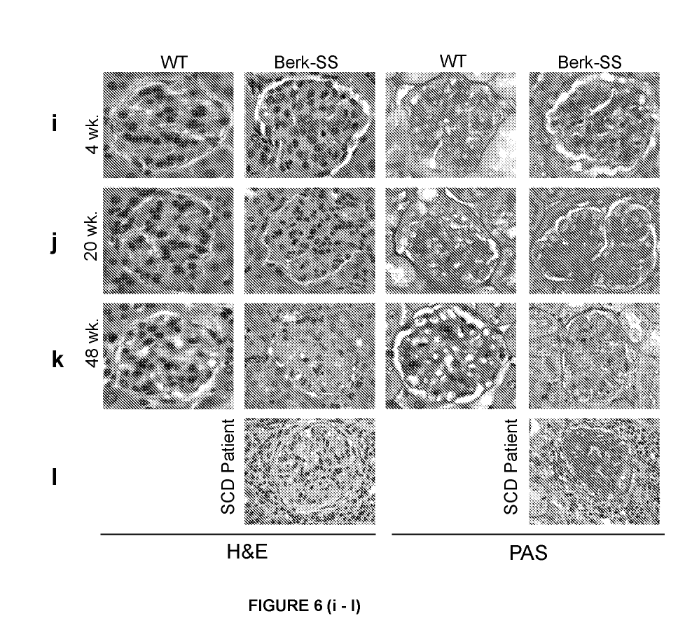

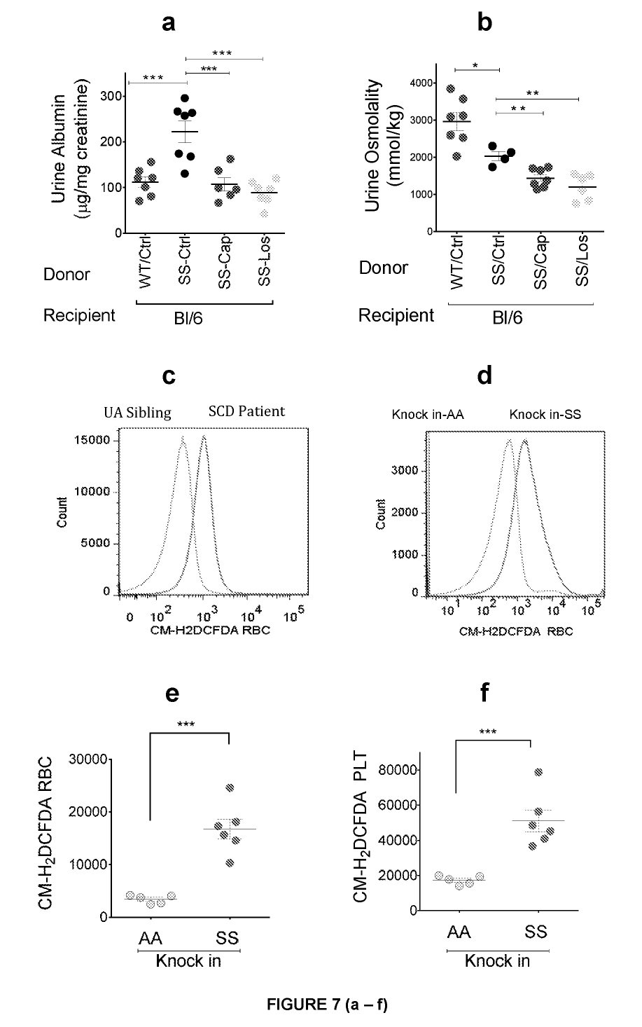

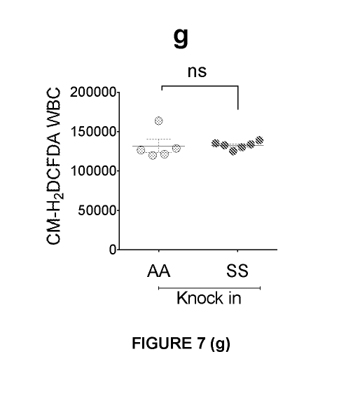

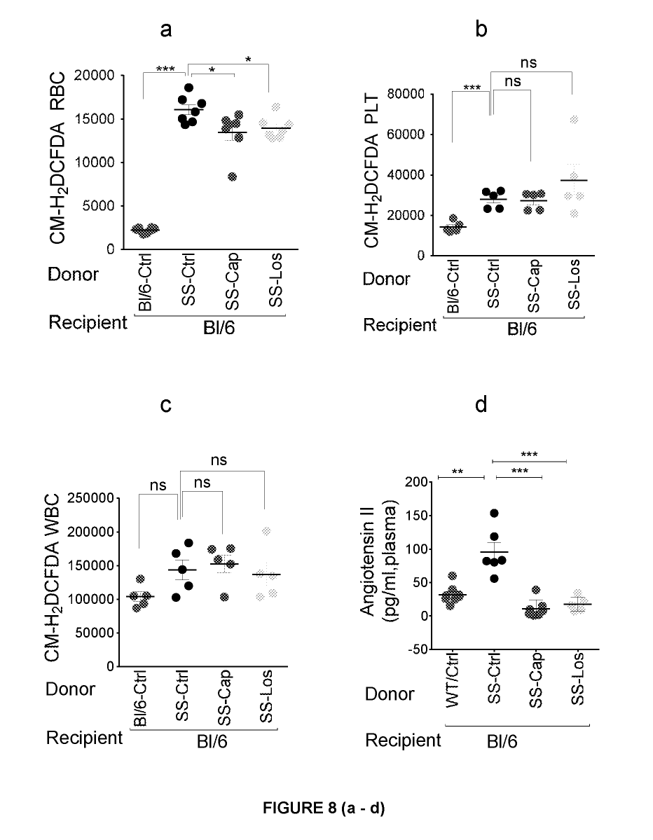

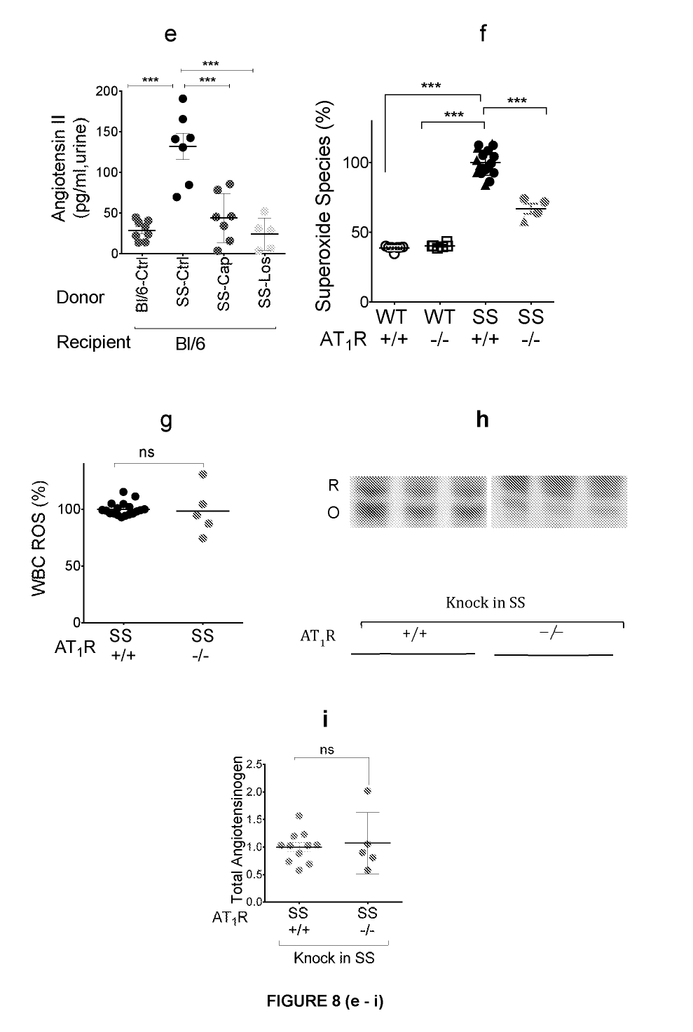

As mentioned herein, the compound N-butyloxycarbonyl-3-(4-imidazol-1-ylmethylphenyl)-5-iso-butylthiophene-2-sulfonamide (C21), with the structure provided in FIG. 1, may be made in accordance with techniques well known to those skilled in the art; for example, as described in international patent application WO 2002/096883, the contents of which are hereby incorporated by reference. In the case of a discrepancy between the name of the compound and the structure provided in FIG. 1, the structure provided in FIG. 1 should prevail.

The skilled person will understand that all embodiments of the invention as described wherein may be combined with one or more other embodiments of the invention. Further, the embodiments described in one aspect of the present invention are not limited to the aspect described. The embodiments may also be applied to a different aspect of the invention as long as the embodiments do not prevent these aspects of the invention from operating for its intended purpose.

All patents, patent applications and publications referred to herein are incorporated by reference in their entirety. In the event of conflicting terminology, the present specification is controlling.

In particular, compounds of the invention may be agonists of the AT2 receptor. More particularly, compounds of the invention may be selective agonists of the AT2 receptor.

Thus, a compound of the invention includes AT2 receptor agonists that fully and those that partially activate the AT2 receptor and those compounds that can stimulate or activate the AT2 receptor. In some embodiments, an AT2 receptor agonist may be defined to include any compound that can stimulate or activate the AT2 receptor. In some embodiments, the compound of the invention is an AT2 receptor specific agonist and binds selectively to the AT2 receptor.

By compounds that “bind selectively” to the AT2 receptor, we include that the affinity ratio for the relevant compound (AT2:AT1) is at least 50:1, for example, at least 100:1, preferably at least 1000:1, more preferably at least 10000:1, and even more preferably at least 25000:1.

According to particular embodiments of the invention, there is provided a method, compound for use or use wherein the angiotensin II receptor agonist is an AT2 receptor agonist or other compound that stimulates an AT2 receptor (e.g. a selective AT2 receptor agonist), or a pharmaceutically acceptable salt, solvate or prodrug thereof.

A particular compound of the invention that may be mentioned is N-butyloxycarbonyl-3-(4-imidazol-1-ylmethylphenyl)-5-iso-butylthiophene-2-sulfonamide (also known as C21).

Thus, according to more particular embodiments of the invention, there is provided a method, compound for use or use wherein the angiotensin II receptor agonist (e.g. the AT2 receptor or other compound that stimulates an AT2 receptor, such as a selective AT2 receptor agonist) is N-butyloxycarbonyl-3-(4-imidazol-1-ylmethylphenyl)-5-iso-butylthiophene-2-sulfonamide, or a pharmaceutically acceptable salt, solvate or prodrug thereof.

As described herein, particular pharmaceutically acceptable salts of compounds of the invention that may be mentioned include the HCl salt.

Thus, for the avoidance of doubt, there is provided a method, compound for use or use wherein the angiotensin II receptor agonist (e.g. the AT2 receptor or other compound that stimulates an AT2 receptor, such as a selective AT2 receptor agonist) is N-butyloxycarbonyl-3-(4-imidazol-1-ylmethylphenyl)-5-iso-butylthiophene-2-sulfonamide, or the HCl salt thereof.

In particular embodiments, compounds of the invention may be referred to as having an anti-nephropathic effect, with a reduction in end organ damage to the kidneys. In particular, compounds of the invention may reduce SCD-associated nephropathy.

Compounds of the invention may be administered either alone or in combination with: other AT2 agonists that are known in the art; AT1 receptor antagonists that are known in the art, such as losartan; and/or inhibitors of angiotensin converting enzyme (ACE) that are known in the art. Such combinations may therefore be useful in the therapeutic treatment of SCD.

Thus, in particular embodiments, there is provided a method, compound for use or use wherein the angiotensin II receptor agonist, or a pharmaceutically acceptable salt, solvate or prodrug thereof, is administered in combination with:

(i) an AT1 receptor antagonist, or a pharmaceutically acceptable salt, solvate or prodrug thereof; and/or
(ii) an inhibitor of angiotensin converting enzyme (ACE), or a pharmaceutically acceptable salt, solvate or prodrug thereof.

In particular, such administration in combination can involve concurrent, prior or subsequent administration of the combination drug with respect to the administration of the angiotensin II receptor agonist.

The compounds of the invention will normally be administered orally, intravenously, subcutaneously, buccally, rectally, dermally, nasally, tracheally, bronchially, by any other parenteral route or via inhalation, in a pharmaceutically acceptable dosage form. Additional methods of administration include but are not limited to intraarterial, intramuscular, intraperitoneal, intraportal, intradermal, epidural, and/or intrathecal administration.

The compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like. Such formulations may be prepared in accordance with standard and/or accepted pharmaceutical practice.

Thus, in a second aspect of the invention, there is provided a method of treatment of SCD, which method comprises administration of a therapeutically effective amount of a pharmaceutical formulation comprising an angiotensin II receptor agonist, or a pharmaceutically acceptable salt, solvate or prodrug thereof, in admixture with a pharmaceutically acceptable adjuvant, diluent or carrier, to a subject suffering from SCD.

In an alternative second aspect of the invention, there is provided a pharmaceutical formulation comprising an angiotensin II receptor agonist, or a pharmaceutically acceptable salt, solvate or prodrug thereof, in admixture with a pharmaceutically acceptable adjuvant, diluent or carrier, for use in the treatment of SCD.

In particular embodiments of the first and second aspects of the invention, administering comprises oral, intravenous, subcutaneous, buccal, rectal, dermal, nasal, tracheal, bronchial, inhalation, intraarterial, intramuscular, intraperitoneal, intraportal, intradermal, epidural, and/or intrathecal administration.

The skilled person will understand that such formulations will comprise a therapeutically effective dose of compounds of the invention.

Depending upon the subject to be treated and the route of administration, the compounds of the invention may be administered at varying doses. Although doses will vary from subject to subject, suitable daily doses (i.e. therapeutically effective doses) are in the range of about 1 to 1000 mg (e.g., 1, 5, 10, 15, 20, 25, 30, 35, 40, 45, 50, 75, 100, 150, 200, 250, 300, 350, 400, 450, 500, 550, 600, 650, 700, 750, 800, 850, 900, 950, 1000 mg, and the like, or any range or value therein) per subject, administered in single or multiple doses. More preferred daily doses are in the range 2.5 to 250 mg (e.g., 2.5, 3, 3.5, 4. 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, mg and the like or any range or value therein) per subject.

Individual doses of compounds of the invention may be in the range 1 to 100 mg (e.g., 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, and the like, or any range or values therein).

In any event, the physician, or the skilled person, will be able to determine the actual dosage which will be most suitable for an individual subject, which is likely to vary with the condition that is to be treated, as well as the age, weight, sex and response of the particular subject to be treated. The above-mentioned dosages are exemplary of the average case; there can, of course, be individual instances where higher or lower dosage ranges are merited, and such are within the scope of this invention.

For the avoidance of doubt, according to particular embodiments, the angiotensin II receptor agonist comprised in the pharmaceutical formulation is an AT2 receptor agonist or other compound that stimulates an AT2 receptor (e.g. a selective AT2 receptor agonist), or a pharmaceutically acceptable salt, solvate or prodrug thereof.

Moreover, according to even more particular embodiments of the invention, the angiotensin II receptor agonist comprised in the pharmaceutical formulation is N-butyloxycarbonyl-3-(4-imidazol-1-ylmethylphenyl)-5-iso-butylthiophene-2-sulfonamide, or a pharmaceutically acceptable salt, solvate or prodrug thereof (such as the HCl salt thereof).

As described herein, compounds of the invention may be administered alone or in combination with certain other active ingredients. The skilled person will understand that combination products (e.g. pharmaceutical formulations) may be prepared that further comprise such active ingredients.

Thus, in a third aspect of the invention, there is provided a method of treatment of SCD, which method comprises administration of a therapeutically effective amount of a combination product (e.g. a pharmaceutical formulation) comprising an angiotensin II receptor agonist, or a pharmaceutically acceptable salt, solvate or prodrug thereof, and:

(A) an AT1 receptor antagonist, or a pharmaceutically acceptable salt, solvate or prodrug thereof; and/or
(B) an ACE inhibitor, or a pharmaceutically acceptable salt, solvate or prodrug thereof,
wherein each of the components is formulated in combination and in admixture with a pharmaceutically-acceptable adjuvant, diluent or carrier, to a subject suffering from SCD.

In an alternative third aspect of the invention, there is provided a combination product (e.g. a pharmaceutical formulation) comprising an angiotensin II receptor agonist, or a pharmaceutically acceptable salt, solvate or prodrug thereof, and:

(A) an AT1 receptor antagonist, or a pharmaceutically acceptable salt, solvate or prodrug thereof; and/or
(B) an ACE inhibitor, or a pharmaceutically acceptable salt, solvate or prodrug thereof,
wherein each of the components is formulated in combination and in admixture with a pharmaceutically-acceptable adjuvant, diluent or carrier, for use in the treatment of SCD.

Such combination be presented either as separate formulations, wherein at least one of those formulations comprises an angiotensin II receptor agonist (as defined herein, e.g., a compound of the invention, or a pharmaceutically acceptable salt, solvate or prodrug thereof), and at least one formulation comprises

(A) an AT1 receptor antagonist, or a pharmaceutically acceptable salt, solvate or prodrug thereof; and/or
(B) an ACE inhibitor, or a pharmaceutically acceptable salt, solvate or prodrug thereof,
or may be presented (i.e. formulated) as a combined preparation (i.e. presented as a single formulation including said components).

Thus, in further aspects of the invention there is provided:

(1) a pharmaceutical formulation comprising an angiotensin II receptor agonist, or a pharmaceutically acceptable salt, solvate or prodrug thereof, and an AT1 receptor antagonist, or a pharmaceutically acceptable salt, solvate or prodrug thereof, and/or an ACE inhibitor, or a pharmaceutically acceptable salt, solvate or prodrug thereof, in admixture with a pharmaceutically-acceptable adjuvant, diluent and/or carrier, for use in the treatment of SCD; and
(2) a kit of parts comprising as separate components:
(a) a pharmaceutical formulation comprising an angiotensin II receptor agonist, or a pharmaceutically acceptable salt, solvate or prodrug thereof, in admixture with a pharmaceutically-acceptable adjuvant, diluent and/or carrier; and
(b) a pharmaceutical formulation comprising an AT1 receptor antagonist, or a pharmaceutically acceptable salt, solvate or prodrug thereof, and/or an ACE inhibitor, or a pharmaceutically acceptable salt, solvate or prodrug thereof, in admixture with a pharmaceutically-acceptable adjuvant, diluent and/or carrier,
which components (a) and (b) are each provided in a form that is suitable for administration in conjunction with the other, for use in the treatment of SCD.

Again, for the avoidance of doubt, according to particular embodiments, the angiotensin II receptor agonist comprised in the combination product (e.g. the formulation or kit of parts) is an AT2 receptor agonist or other compound that stimulates an AT2 receptor (e.g. a selective AT2 receptor agonist), or a pharmaceutically acceptable salt, solvate or prodrug thereof.

Moreover, according to even more particular embodiments of the invention, the angiotensin II receptor agonist comprised in the combination product (e.g. the formulation or kit of parts) is N-butyloxycarbonyl-3-(4-imidazol-1-ylmethylphenyl)-5-iso-butylthiophene-2-sulfonamide, or a pharmaceutically acceptable salt, solvate or prodrug thereof (such as the HCl salt thereof).

In particular embodiments of the invention (e.g. particular embodiments of each aspect of invention as described herein), the treatment of SCD is in a subject who is not being treated for graft-versus-host disease (GVHD).

In particular embodiments of the invention (e.g. particular embodiments of each aspect of invention as described herein), the treatment of SCD with compounds of the invention is in a subject who is not provided transplant or transfusion therapy (such as a bone marrow transplant, bone marrow transfusion and/or blood transfusion, e.g. for the treatment of SCD) during the 12 months preceding treatment with compounds of the invention, during the treatment with compounds of the invention or during the 12 months following the end of the treatment with compounds of the invention.

In more particular embodiments of the invention (e.g. particular embodiments of each aspect of invention as described herein), the treatment of SCD with compounds of the invention is in a subject who is not provided transplant or transfusion therapy (such as a bone marrow transplant, bone marrow transfusion and/or blood transfusion, e.g. for the treatment of SCD) during the 6 months (particularly, 3 months) preceding treatment with compounds of the invention, during treatment with compounds of the invention or during the 6 months (particularly, 3 months) following the end of the treatment with compounds of the invention.

With reference to the embodiments described above, the skilled person will understand that references to preceding and following periods of time will refer to periods of time preceding or following the time of treatment (or intended treatment) with compounds of the invention.

As described herein, the compounds of the invention are useful because they possess pharmacological activity. In particular, the compounds of the invention are angiotensin II receptor agonists, more particularly, they are agonists of the AT2 receptor, and, especially, are selective agonists of that sub-receptor. Compounds of the invention have the advantage that they bind selectively to, and exhibit agonist activity at, the AT2 receptor.

The compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties than compounds known in the prior art. Such effects may be evaluated clinically, objectively and/or subjectively by a health care professional, a treatment subject or an observer.

Without wishing to be bound by theory, it is thought that increased oxidative stress in SCD results in oxidation of angiotensinogen, which increased its conversion to angiotensin-II. Consequently, significant hyperangiotensinemia has been seen in humans and mice with SCD.

### FIGURES

The invention will now be described in more detail by reference to the following, non-limiting, figures.

FIG. 1: The structure of N-butyloxycarbonyl-3-(4-imidazol-1-ylmethylphenyl)-5-iso-butylthiophene-2-sulfonamide (C21).

FIG. 2: Hyperangiotensinemia Promotes UCA and Glomerulopathy in Sickle Mice by AT1R Mediated TGFβ1 activation and Smad 2/3 phosphorylation.

Representative western blots showing: (a) activated form of TGFβ1 in the Berk-SS glomeruli compared to WT controls. (b) Phosphorylated Smad-2/3 and total Smad2/3 expression in glomerular preparations of WT and Berk-SS kidneys. (c) Graph showing the progression of albuminuria (Y-axis) with weeks of drug treatment (X-axis) in WT mice, untreated Berk-SS control mice, or Berk-SS mice treated with captopril or losartan. Mice were started on drug treatment at 4 weeks of age (d-g) Hematoxylin-eosin staining (d), PAS staining (e) and immunohistochemistry for phosphorylated Smad-2/3 (f) and Nitrotyrosine (g) in kidneys of WT mice, untreated Berk-SS control mice, or Berk-SS mice treated with captopril or losartan. (h) Graph of progression of UCA, measured by urine osmolality (Y-axis) in WT mice (dark red), untreated Berk-SS control mice (black), or BerkSS mice treated with captopril (blue) or losartan (green) with weeks of drug treatment depicted on the X-axis. Mice were started on drug treatment at 4 weeks of age. Urine osmolality could not be measured in most mice on captopril, due to high mortality from severe hyposthenuria and dehydration. (i) Kaplan-Meier survival curve in WT mice (dark red line), untreated Berk-SS control mice (black line), or Berk-SS mice treated with captopril (blue line) or losartan (green line) during drug treatment (X-axis). The percentage of mice surviving at the end of the experiment is indicated against the survival curve of each group. Results represent means±S.E.M. Statistical analysis comparing untreated Berk-SS mice to other groups was done using ANOVA (Dunnet's multiple comparisons test). Survival curves were compared using Log Rank test. Statistical significance is denoted by * P<0.05, **P<0.01, ***P<0.001, ****P<0.0001. Data are from three independent experiments.

FIG. 3: Sickle Hematopoiesis and the Role of AT1R and AT2R in UCA and ROS Production.

(a) Plasma angiotensin-II levels in WT and SCD hematopoietic chimeras. WT mice (BI/6; recipient) were transplanted with Berk-SS (SS) or WT (BI/6) donor bone marrow and mice were determined to be fully chimeric for donor bone marrow are shown. (BI/6/BI/6 chimeras n=8, SS/BI/6 chimeras n=6). (b) Urine albumin levels in WT (BI/6), AT1R^{-/-} and AT2R^{-/-} recipient mice transplanted with WT (BI/6; dark red bars) or Berk-SS (SS; black bars) donor bone marrow. Mice fully chimeric for WT or SS bone marrow were analysed for urine albumin levels. (n=6-20 mice per group) (c) Representative H&E and PAS staining of kidneys of SS/BI/6, SS/AT1R^{-/-} and SS/AT2R^{-/-} (donor/recipient) chimeras. Glomerular pathology including glomerulosclerosis and mesangial proliferation and basement membrane thickening is observed in SS/BI/6 and SS/AT2R^{-/-} chimeras, but ameliorated in SS/AT1R chimeras (d) Immunohistochemistry of kidneys of SS/BI/6 and SS/AT1R^{-/-} (donor/recipient) chimeras showing nitrotyrosine, active TGF-β1, and PSmad-2/3 expression. (e) Urine osmolality in WT (BI/6), AT1R^{-/-} and AT2R^{-/-} recipient mice transplanted with WT (dark red bars) or Berk-SS (black bars) donor bone marrow. Mice fully chimeric for WT or SS bone marrow were analysed for urine osmolality after an 8 hour water deprivation. (n=5-9 mice per group). (f-g) Urine osmolality (f) and urine albumin (g) in Berk-SS mice that received AT2R agonist, C21 together with blockade of AT1R signalling with losartan (LOS) or LOS alone. (h) Representative CM-H₂₋DCFDA ROS labelling in Berk-SS (red histogram) or WT (blue histogram) erythrocytes (RBC), showing a higher mean fluorescence in Berk-SS RBC. (i-k) Cumulative data on the mean fluorescence intensities of CM-H₂-DCFDA in red blood cells (RBC), platelets (PLT), and white blood cells (WBC) in WT and Berk-SS mice (n=9 each) (l-n) Mean fluorescence intensities of CM-H₂-DCFDA in RBC, PLT and WBC in SCD patients and their unaffected sibling controls (Unaffected siblings n=12, SCD patients n=9). All data is plotted as means±SEM. Statistical analysis comparing Berk-SS and WT mice, or SCD patients and controls was done using unpaired t-tests, ANOVA (Dunnet's multiple comparisons test) was used while comparing between multiple groups. Statistical significance is denoted by * P<0.05, **P<0.01, ***P<0.001, ****P<0.0001.

FIG. 4: ROS-mediated hyperangiotensinemia is initiated by sickle erythrocytes and perpetuated by AT1R-mediated ROS generation from erythrocyte NADPH oxidase.

(a-b) Mean fluorescence intensity of CM-H₂-DCFDA labeled erythrocytes (RBC) (a) and platelets (PLT) in WT control (BI/6-Ctrl) mice, Berk-SS control mice (SS-Ctrl) or Berk-SS mice treated with losartan (SS-Los) or captopril (SS-Cap) (n=4-8/group). (c) Graph of the ratio of oxidized (O) to reduced (R) plasma angiotensinogen in BI/6-Ctrl, SS-Ctrl, SS-Los or SS-Cap mice, determined from the quantified intensities of the respective bands in western blot analysis (n=410/group). (d) Plasma angiotensin levels in in BI/6-Ctrl, SS-Ctrl, SS-Los or SS-Cap mice (n=513/group). Each symbol represents and individual mouse. (e-f) Graph of the relative mean fluorescence intensity of CM-H₂-DCFDA in the RBC (e) or PLT (f) in WT mice (WT/AT1R^{+/+}, black open circles), WT/AT1R^{-/-} mice (red open circles), Berk-SS/AT1R^{+/+} (black triangles) and Knock-in SS/AT1R^{+/+} (black circles), Berk-SS/AT1R^{-/-} (red triangles) and Knock-in SS/AT1R^{-/-} (red triangles) mice (n=7-24/group). (g) Rac-GTP pull down western blot showing WT AT1R^{-/-} and Knock-in SS/AT1R^{-/-} erythrocytes had very low Rac activity basally (at 0 min), or after 5-15 minutes after stimulation with Ang-II. Knock-in SS/AT1R^{+/+} erythrocytes had much higher Rac activation at baseline than WT/AT1R^{+/+} erythrocytes. (h) Graph of the ratio of oxidized (O) to reduced (R) plasma angiotensinogen in Knock-in SS/AT1R^{+/+} and Knock-in SS/AT1R^{-/-} mice, determined from the quantified intensities of the respective bands in western blot analysis. Each symbol represents an individual mouse (n=5-6 mice/group). (i) Urine albumin levels and (j) urine osmolality in Knock-in SS/AT1R^{+/+} and Knock-in SS/AT1R^{-/-} mice. (k-m) Mean fluorescence intensity of CM-H₂-DCFDA labeled erythrocytes (RBC) (k), urine osmolality (l) and urine albumin (m) in Berk-SS/AT1R^{f/f}/EpoR-Cre⁽⁺⁾ mice compared to Berk-SS/AT1R^{f/f}/EpoR-Creo⁽⁻⁾ controls. All data is plotted as means±SEM. Statistical analysis was done either using unpaired t-tests where two groups are compared or using ANOVA (Dunnet's multiple comparisons test) while comparing between multiple groups. Statistical significance is denoted by * P<0.05, **P<0.01, ***P<0.001, ****P<0.0001. (n) Model of ROS-induced hyperangiotensinemia in SCD with its beneficial effects (shown in blue colour) and harmful effects (shown in red) in SCD pathophysiology. High oxidative stress in SCD causes hyperangiotensinemia. Sickle erythroid cells generate high amounts of ROS (red) via angiotensin receptor 1 AT1R signaling, necessary to enhance erythropoiesis (blue). Increased erythroid ROS further induces hyperangiotensinemia (red) by oxidation of its precursor as a positive feedback loop. Hyperangiotensinemia, in turn signals through AT1R and AT2R in the kidneys to improve UCA (blue) but also mediates end organ damage, such as glomerulopathy via increased TGFβ1 production (red). Indeed, increased AT1R-TGFβ1 signaling may be an important mediator of end organ damage in the heart, lung and blood vessels in SCD.

FIG. 5. Renal parameters associated with hyperangiotensinemia in humans and mice with SCD.

(a) SCD patients with hyperangiotensinemia (shown in FIG. 5 a-b) have normal systolic (sBP) and diastolic (dBP) blood pressures. Not shown is that these SCD patients showed no evidence of albuminuria (urine microalbumin 18±4.6 mg/g creatinine) and other renal parameters (BUN, serum creatinine). (b) Plasma renin concentrations in young (<24 week old) and old (>24 week old) SCD and WT mice. Plasma renin was significantly higher only in old Berk-SS mice. (c-d) Representative western blot analysis showing renin expression in the glomeruli of young and old WT and Berk-SS mice. Quantification of the intensity of the bands in the western blots showed a trend towards higher renin expression in old sickle mice. (e) Higher plasma oxidized angiotensinogen (ANG) was seen in human patients with SCD compared to their unaffected siblings. R: reduced, O: oxidized. (f) Western blot analysis of nitrotyrosine expression in Berk-SS kidneys as compared to WT kidneys. Plotted data is represented as mean±SEM. Statistical analysis was done using ANOVA (Dunnet's multiple comparisons) or unpaired t-tests. Statistical significance is denoted by * P<0.05, **P<0.01, ***P<0.001, ****P<0.0001.

FIG. 6. Berk-SS and Knock-in SS mice served as a robust model of human SN.

(a) Urine osmolality of WT, Berk-SS mice, Knock-in AA and Knock-in SS mice on a 24 hour urine collection (n-13-33/group), showing that both types of SCD mice have a lower UCA. (b and e) WT (C57BI/6) mice had similar and normal urine osmolality and normal urine albumin levels as Berk-AA mice, while Berk-SS mice showed low UCA and albuminuria, classic of SN. Since albuminuria and osmolality values for Berk-AA and C57BI/6 mice were similar, either of them were used as WT controls (n=15-25 for Berk-AA, Berk-SS or C57BI/6 mice). (c) Water deprivation significantly increases UCA in WT mice. However, 8-hour water deprivation was only possible in WT mice and resulted in nearly ˜80% mortality in Berk-SS mice. Hence 24-hour urine was collected without water deprivation (n=9-22 mice per group) to determine UCA. (d) Urine albumin (normalized to urine creatinine) in WT, Berk-SS mice, Knock-in AA and Knock-in SS mice (n-14-58/group) of mice shows that SCD mice develop significant albuminuria. (f) Temporal analysis showed that urine albumin values in Berk-SS mice significantly increased by young adulthood (8 weeks of age) and plateaued thereafter. WT mice had the same level of albuminuria through all the ages analyzed. (WT mice, n=20, Berk-SS mice: 4 week-old n=10, 8 week-old n=6, 12 week-old n=14 and 24 week-old n=25). (g) Berk-SS mice have a high mortality resulting in survival of the fit with increasing age. (h) Glomerular filtration rate (GFR) in WT and Berk-SS mice reproduced the GFR pattern as seen in children and adults with SCD. (WT mice 4 weeks n=3, 8-10 weeks n=5, 24 weeks n=2 Berk-SS 4-8 weeks n=4, 16-20 weeks n=2). (i-k) Representative histological (H&E and PAS) analysis of glomeruli of young and old WT and Berk-SS mice. The majority of glomeruli showed mesangium and capillary loops that were variably thickened by accumulations of an eosinophilic, amorphous to fibrillar material with narrowing or obliteration of capillary lumina (glomerulosclerosis) which advanced with age. Tubules were minimally affected. (l) Kidney sections of a SCD patient with macroalbuminuria shows similar advanced FSGS pattern as seen in Berk-SS mice. Data are from six independent experiments. Data in all four panels is represented as mean±SEM. Statistical analysis comparing Berk-SS and WT mice and Knock-in AA and Knock-in SS mice was done using unpaired t-tests, while other graphs were done using ANOVA (Dunnet's multiple comparisons test). Statistical significance is denoted by *P<0.05, **P<0.01, ***P<0.001, ****P<0.0001.

FIG. 7. ROS, hyperangiotensinemia and AT 1 R mediated SN in sickle hematopoietic chimeras and Knock-in sickle mice.

BM from Berk-SS mice or WT (BI/6) mice was transplanted into BI/6 mice; only mice fully chimeric for sickle hematopoiesis were analysed for nephropathy. (a) Berk-SS/WT chimeras developed albuminuria, in contrast to WT/WT chimeras. Berk-SS/WT chimeric mice placed on captopril or losartan did not develop albuminuria compared to the untreated Berk-SS/WT chimeric mice (b) AT₁R blockade with losartan and captopril worsened UCA in sickle chimeric mice. Urine osmolality was determined after an 8 hour water deprivation (which was better tolerated by Berk-SS/WT chimeric mice, in contrast to Berk-SS mice; (n=6-8/group). (c) Representative flow cytometry histogram following CM-H₂DCFDA staining showing higher RBC ROS in SCD patients compared to unaffected siblings. (d) Representative flow cytometry histogram following CM-H₂-DCFDA staining showing higher RBC ROS in Knock-in SS mice than Knock-in AA mice. (e-g) Cumulative data on the mean fluorescence intensities of CM-H₂-DCFDA labelling in RBC, platelets and WBC in Knock-in AA and SS mice (n=3 each). While RBC and platelets (PLT) show higher ROS than AA mice, WBC ROS is similar between them. Data in all panels represented as mean±SEM. Statistical analysis was done using ANOVA (Dunnet's multiple comparisons) or unpaired t-tests. Statistical significance is denoted by *P<0.05, **P<0.01, ***P<0.001, ****P<0.0001.

FIG. 8. ROS, ANG and Ang-II in sickle hematopoietic chimeras and AT₁R deficient SCD mice.

(a-c) ROS levels in RBC, PLT and WBC in control and drug treated Berk-SS chimeras showing that captopril and losartan treatment significantly lowered ROS specifically in RBC, not platelets. (B6/B6 n=8, SS/B6 n=7, SS/B6 on captopril n=7 and SS/B6 on losartan n=7). (d-e) Plasma and urine AngII levels are lower in both captopril and losartan treated mice compared to untreated Berk-SS controls (n=5-13/group). (f) The superoxide species among WT/AT₁R^{-/-} and WT/AT₁R^{+/+} mice are not different from each other. However, the superoxide species is significantly lower in Knock-in SS/AT₁R^{-/-} mice compared to Knock-in SS/AT₁R^{+/+} mice. (g) The percent WBC ROS in Knock-in SS mice deficient in AT₁R is similar to those with AT₁R. (h) Western Blot showing the oxidized:reduced status of ANG in the plasma of Knock-in SS/AT₁R^{-/-} mice compared to Knock-in SS/AT₁R^{+/+} showing a reversal of the oxidized:reduced ANG ratio R: reduced, O: oxidized. (i) The total ANG among these two groups remained unchanged. Graph comparing the intensities of the western blots for total ANG for the above two groups. Data represented as mean±SEM. Statistical analysis was done using ANOVA (Dunnet's multiple comparisons test). Statistical significance is denoted by *P<0.05, **P<0.01, ***P<0.001, ****P<0.0001.

### Materials and Methods:

Mice:

Berkeley SCD mice (Berk-SS)(Hba^{tm1Paz} Hbb^{tm1Tow} Tg(HBA-HBBs)41Paz/J were primarily used as the sickle cell disease model in this study. Mouse α- and β-globin genes are knocked out in the Berkeley sickle mice while a transgene for the human α- and β^{s}-globin genes (SS) is introduced into their genome. Their normal counterparts (Berk-AA) have the transgene carrying normal β-globin (AA) instead of β^{s}-globin and were kindly provided by Dr. Cheryl Hillar (Madison, Wis.). The Berk-SS mice were derived from four different genetic strains of mice and backcrossed to C57 BI/6 mice for 6 generations. Hence, they can be used as donors for transplant into BI/6 mice. The Berk-AA mice are still relatively outbred and cannot be transplanted into BI/6 or Berk-SS mice without significant graft versus host disease. Knock-in SS (B6; 129-Hba^{tm1(HBA)Tow} Hbb^{tm2(HBG1,HBB*)Tow}/Hbb^{tm3(HBG1,HBB)Tow}/J) mice were kindly provided by Dr. Timothy Townes (University of Alabama, Birmingham). AT₁R^{-/-} mice (B6.129P2Agtr1a^{tm1Unc}/J) were purchased from Jackson laboratories (Bar Harbor, Me.). AT₂R^{-/-} mice were kindly provided by Dr. Tadashi Inagami (Vanderbilt University School of Medicine, Nashville, Tenn.). Bone marrow from donor Berkeley sickle mice (or 057BI/6 control mice) were distributed 1:5 among the 057BI/6, AT₁R^{-/-} and AT₂R^{-/-} lethally irradiated (1175 cGy) recipient mice. All animals were maintained in the Cincinnati Children's Research Foundation's vivarium using protocols approved by the Institutional Animal Care and Use Committee.

Urine Collection, Urine Albumin and Osmolality:

Twenty-four hour urine was collected using metabolic cages. Urine volume was measured and then protein stabilization buffer (0.4 mM ophenanthroline, 1 mM p-hydroxymercuribenzoic acid and 0.12 mM pepstatin A and 0.5M EDTA, pH 8.0) was added to a portion of urine to stabilize Ang-11. The rest of the urine was used for other analyses, including GFR, albumin, osmolality. Temporal assessments of urine albumin, creatinine and osmolality were made after an 8 hour water deprivation in SCD chimeric mice. Berk-SS mice did not tolerate 8 hour water deprivation, which caused very high mortality from dehydration. Therefore, in Berk-SS mice (untransplanted/“straight SCD” native sickle mice) no water deprivation was performed prior to urine collection. Urine albumin level was measured using the mouse albumin ELISA kit (Bethyl Laboratories, Montgomery, Tex., catalog# E90-134). The ELISA was performed in a 96-well clear micro-plate (R&D systems, Minneapolis, Minn., catalog # DY990) as per manufacturer's instructions. Urine osmolality was measured by vapor pressure osmometer, Vapro 5600, (Wescor Biomedical Systems, South Logan, Utah). Urine creatinine was measured using the creatinine parameter assay kit (R&D Systems, Minneapolis, Minn., catalog# KGE005). Plasma creatinine was measured using the creatinine assay kit (Abcam, Cambridge, Mass., catalog# ab65340) following the manufacturer's instructions.

ROS Analysis:

ROS was measured in WBC, RBC and platelets by mixing 0.5 μl of whole blood with 100 μl of FACS buffer (1×PBS, 0.5% bovine serum albumin), 0.1 μl of biotin anti-mouse Ter-119/erythroid Ly-76 antibody (BD Biosciences, San Jose, Calif., catalog#553672), 1 μl of Streptavidin APC-CyTM⁷ (BD Biosciences, catalog#554063), 1 μl of PE anti-mouse CD45 antibody (BD Biosciences, cat#553081). Four compensation tubes included unstained, biotin anti-mouse Ter-119 along with Streptavidin APC-CyTM⁷, PE anti-mouse CD45 and a CM-H₂DCFDA (5-[and-6]-carboxy-2′,7′-dichlorofluorescein diacetate (Life Technologies, Grand Island, N.Y., catalog# C6827)) were concurrently prepared. All the tubes were incubated at room temperature for 20-30 minutes. The samples were washed with PBS at 200×g for 5 min and 100 μl of CM-H₂DCFDA in 1:200 dilution was added to all sample tubes and the CM-H₂DCFDA compensation tube while 100 μl of PBS was added to the rest of three compensation samples. The tubes were incubated for 30 minutes at 37° C. All the tubes were washed with 500 μl of PBS and centrifuged at 200×g for 5 min. After removing the supernatant the pellet was re-suspended in 200 μl of ice-cold PBS. Samples were stored on ice until analyzed on a BD FACSCanto™ II flow cytometer (BD Biosciences, San Jose, Calif.) by gating on the WBC, RBC and platelets.

ROS/Superoxide/RNS Assay:

Reactive oxygen and nitrogen species (ROS/RNS) production in whole blood was measured by following the ROS/RNS detection kit (Enzo Life Sciences Inc., Farmingdale, N.Y., catalog#ENZ-51001-200 & ENZ-51010) protocol. Briefly, 1 μl of whole blood from experimental animals were mixed with ROS/RNS 3-plex detection mix and incubated for 20 minutes at 37° C. Prior to this step positive control were set up by adding nitric oxide inducer (L-Arginine) and ROS inducer (Pyocyanin) to whole blood in separate tubes and incubating for 30 minutes at 37° C. Along with this negative control tubes were also set up by adding NO scavenger (c-PTIO) and ROS inhibitor (N-acetyl-L-cysteine) and incubating at similar conditions. After incubation the samples were washed with wash buffer at 200×g for 5 min. NO detection reagent (red), oxidative stress detection reagent (green) and superoxide detection reagent (orange) were added separately to positive and negative control tubes as compensation controls and incubated further for 15-20 minutes at 37° C. All samples were kept on ice and analyzed on a BD FACSCanto™ II flow cytometer.

Angiotensinogen Redox and Angiotensin II Analysis:

To measure the redox status of angiotensinogen, 5 μl of freshly separated plasma was mixed with 5 μl reaction buffer (100 mM Tris-HCl, pH 8.0, 5 mM EDTA, 0.15M NaCl) and 10 μl of 20 mM polyethylene glycol adduct PEG5000 maleimide, termed mPEG5K, Sigma-Aldrich, St. Louis, Mo., catalog#63187-1G) was incubated for 3 hrs at 37° C. To this, 80 μl of 1× Laemmli sample buffer was added and the sample stored at −80° C. Western blot was performed by loading 20 μl of this plasma preparation on a 10% Mini-PROTEAN® TGX™ precast gel (BIO-RAD, Hercules, Calif., catalog#456-1033) and subjected to electrophoresis (at 200V for 40 minutes) and transferred on to a PVDF membrane. The membrane was stained with Ponceau S for 1 hr to estimate protein loading. Ponceau S was removed by washing twice for 15 min with PBS, and blotted with an antiangiotensin (N-10) antibody (Santa Cruz Biotechnology, Inc, Santa Cruz, Calif., catalog# sc-7419), secondarily labeled with rabbit anti-goat IgG, horseradish peroxidase (HRP) conjugate (Life Technologies, Grand Island, N.Y., catalog# R-21459). The oxidized and reduced band intensity was measured using Image J software (National Institutes of Health, Bethesda, Md.) and plotted using GraphPad Prism software (GraphPad Software Inc., La Jolla, Calif.).

Urine and plasma Angiotensin-II level was measured by using the Angiotensin II EIA kit (Cayman Chemical Company, Ann Arbor, Mich., catalog# A05880) following the manufacturer's instructions.

Western Blots:

Protein was loaded on to 10% Mini-PROTEAN® TGX™ pre-cast gels and electrophoresed at 200V for 40-45 min, then transferred on to a PVDF membrane at 100V for 1 hr or 30V overnight in cold room (4° C.) with constant stirring. The membrane was blocked in buffer containing TBST (1×TBS+0.1% Tween20), 2.5% BSA and 0.025% NaN₃ for 1 hr. Required dilutions of primary antibodies (listed below) were prepared using the above block solution and the blot incubated overnight immersed in the primary antibody solution at 4° C. on a rocking platform. The blot was washed the following day with TBST (thrice for 8 min each) and probed with secondary antibody-horse radish peroxidase conjugate was prepared in the block solution, and added for 4-6 hrs at 4° C. on a rocking platform. Blot was then washed (thrice for 8 min each time) and an ECL substrate (Thermo Scientific, Florence, Ky., catalog#32106) was added to bind to the secondary antibody horseradish peroxidase conjugate. The blot was developed using a LAS-1000 imaging system (FujiFilm, Edison, N.J.). Total kidney protein lysate (50 μg) was loaded per lane for nitrotyrosine and AT1R detection; 22 μg of glomerular extract was loaded per lane for the detection of active TGF-β, Phospho-smad2/3 and total psmad2/3; 2 μl packed RBCs were loaded per well for detection of AT1R protein expression.

The following antibodies were used for western blots: Nitrotyrosine antibody (R&D systems, Minneapolis, Minn., catalog# MAB3248), anti-Angiotensin (N-10) antibody (Santa Cruz biotechnology, Santa Cruz, Calif., catalog# sc-7419); anti-goat HRP secondary antibody (Life Technologies, Grand Island, N.Y., catalog# R-21459); TGF-β pan specific polyclonal Ab (R&D Systems, Minneapolis, Minn., catalog# AB-100-NA); anti-GAPDH antibody [6C5] (Abcam, Cambridge, Mass. catalog# AB8245); phospho-smad2 (ser465/467)/smad3 (ser423/425) (D6G10) (Cell Signaling, Danvers, Mass. catalog#9510); anti-smad2, phospho-specific (ser465/467) (Millipore, Billerica, Mass., catalog# AB3849); anti-smad2/3 (Millipore, Billerica, Mass. catalog#07408); anti-rabbit AT1R antibody (Alomone labs, Jerusalem, Israel, catalog# AAR-011), stabilized rabbit anti-mouse HRP conjugated antibodies (Thermo Scientific, Rockford, Ill., catalog#31456); stabilized goat anti-rabbit HRP conjugated antibodies (Thermo Scientific, Rockford, Ill., catalog#31460).

Whole Kidney and Glomerular Protein Isolation:

One kidney was transferred to a 5 ml polystyrene round-bottom tube (Becton, Dickinson and Company, Franklin Lakes, N.J., catalog#352235) containing 2 ml of ice-cold 1× protein lysis buffer (Tris/HCl, pH 8.0, 20 mM, NaCl 0.14M, EGTA 1 mM, glycerol 1%, MgCl₂ 1.5 mM, 1 mM sodium vanadate, 50 mM sodium fluoride (NaF), protease inhibitor tablet (complete ultra tablets, mini, Roche Applied Science, Indianapolis, Ind., catalog#05892970001). The kidney was homogenized using a tissue homogenizer on ice. The homogenized solution was transferred to 1.7 ml tubes and centrifuged at 16,000×g, at 4° C. for 1 hr. The supernatant was transferred to a fresh tube and a centrifuged once more at 10,000×g at 4° C. for 10 minutes The supernatant from the second spin was transferred to a fresh tube and protein was quantified by Bradford assay (BIO-RAD, Hercules, Calif., catalog#500-0006). For glomerular protein, 75-100 μl of the protein lysis buffer was added to the frozen glomerular pellets in 1.7 ml tubes and dissolved by pipetting up and down several times. The tubes were kept on ice for 10 minutes. They were then sonicated by pulsing at 40 volts for 5 seconds three times at 4° C. In between sonication the tubes were kept on ice to cool down the heat generated due to sonication. The tubes were then centrifuged at 8000×g, at 4° C. for 10 minutes. The supernatant was transferred to fresh tubes and protein was quantified by Bradford assay.

Glomeruli Isolation:

Kidneys were isolated in ice-cold PBS and kept cold on ice and RNAsefree equipment and plastic-ware in an RNase free area during the procedure. The kidneys were minced with a fresh clean razor blade into a watery consistency. They minced kidney suspension was transferred into 1.7 ml tubes containing 500 μl of 1% collagenase. Tubes were then incubated at 37° C. for 30 minutes in a thermo-mixer with constant stirring. The tubes were triturated vigorously by pipetting few times every minute during the incubation. After 30 minutes of incubation, 1 ml of ice-cold 5% FBS in 1×PBS was added to the tubes and filtered through a 100 μM filter, with filtrate collected in a 50 ml tube. The filtrate was then further passed through a 40 μM filter to capture the glomeruli. The 40 μM filter was rinsed with 0.1% FBS in PBS, and then flipped over a 6 cm dish and 0.1% FBS in 1×PBS was passed through the opposite side of the filter. Glomeruli were captured in the in the 6 cm dish. The glomerular solution was then centrifuged at 500×g for 10 mins at 4° C. The glomerular pellet was re-suspended in 1.5 ml 0.1% FBS/PBS, and centrifuged at 500×g at 4° C. for 10 min. The pellets were flash frozen in liquid nitrogen and stored at −70° C. for future protein analysis.

Histology and Immunohistochemistry:

Slides made from paraffin embedded blocks of mouse kidney were de-paraffinized in xylene (thrice for 5 min each) and then hydrated in ethanol ETOH transferring them from higher to lower alcohol ethanol concentrations (100%, 95% and 70% ethanol for 2 min each). After briefly rinsing with distilled water the slides were boiled in 10 mM sodium citrate (pH 6.0) for 10 min, cooled at room temperature for 30 min and rinsed in distilled water. Using a hydroscopic PAP pen the area of interest was bordered. Slides were then rinsed in PBS and incubated in hydrogen peroxide for 10 min. After rinsing the slides in PBS (thrice for 5 min each), the slides were blocked (10% normal goat serum+0.3% TritonX-100 in PBS) for 1 hr at room temperature (RT). Primary antibodies diluted in block solution at a 1:200 (p-smad2/smad3, TGF-β) and 1:150 (nitrotyrosine) concentrations were added to sections and incubated overnight at 4° C. The next day the slides were rinsed with PBS (thrice for 5 min each time), and were incubated in biotinylated goat anti rabbit secondary antibody (psmad2/smad3) and biotinylated goat anti mouse secondary antibody (nitrotyrosine) (diluted in 1:200 in 10% normal goat serum+0-3% triton X-100) for 1 hr at RT. The slides were then rinsed in PBS (thrice for 5 min each). Avidin-biotin-complex was made by using Vector Lab's ELITE kit (Vector Laboratories Inc, Burlingame, Calif. catalog# PK-6200). One drop of solution A was mixed with 1 drop of solution B into 10 ml of PBS, vortexed; slides were incubated in avidin-biotin complex for 1 hr at RT. The slides were rinsed in PBS (thrice for 5 minutes each time) and then in water (twice for 2 minutes each). DAB (3, 3′-diaminobenzidine) solution was made by using DAB Peroxidase Substrate Kit, 3,3′-diaminobenzidine (Vector Laboratories Inc. Burlingame, Calif., catalog# SK-4100), using 5 ml of distilled water, 2 drops of buffer, 4 drops of 3, 3′diaminobenzidine, 2 drops of hydrogen peroxide solution, and vortexed. Slides were incubated with 3, 3′-diaminobenzidine for 5 min at RT while protected from light, rinsed in PBS (twice for 5 min) and then rinsed in distilled water (twice for 2 min). For nuclear staining the slides were immersed in Hematoxylin solution for 45 seconds. They were then dehydrated at 70% ethanol twice for 5 min, 95% ethanol twice for 5 min and in 100% ethanol thrice for 5 min and then cleared in Xylene thrice for 5 min, and mounted in histomount.

Masson's trichrome staining, H&E staining; PAS staining were done by the Cincinnati Children's Hospital Pathology Core Facility. All the staining procedures were performed using the Ventana Symphony staining platform (Ventana Medical Systems, Tucson Ariz.).

Rac-GTP Pull Down Assay:

Erythrocytes were washed 3 times with PBS and spun at 100×g for 3 min in a microfuge. 50 μl of packed erythrocytes per mouse sample were used. Samples were incubated overnight at 4° C. with PBS on a rocker. The samples were stimulated with 2 μM Angiotensin in 100 μl of total volume (with PBS) for 5 min, 20 min and 4 hrs. They were incubated at 37° C. for the different time points. The samples were diluted with 700 μl of PBS to stop the stimulation and then spun down and snap frozen. Cells were thawed on ice when ready to use and sonicated 3 times for 5 sec, spun down and the supernatant was used. Rac was pulled down using the Millipore Products and protocol.

Drugs:

Mice were given the following drug treatments: a) Captopril (West-Ward Pharmaceutical, West Eatontown, N.J., NDC number #0143-1173-01) 0.15 mg/ml in drinking water. b) Losartan (Teva Pharmaceuticals USA Inc, North Wales, Pa., -NDC number#00937366-98) 0.3-0.6 mg/ml in drinking water. c) C21 (Vicore Pharma AB, Haraldsgatan 5, S-413 14 Goteborg, Sweden; Batch/Lot number # A11202910) 10 mg/kg/day in drinking water. Losartan and captopril water bottles were changed twice a week with fresh drugs and C21 was changed daily.

Renin ELISA: Plasma renin was measured following the Mouse Renin 1 ELISA Kit from RayBiotech (RayBiotech, Inc, Norcross, Ga.; catalog# ELM-Renin1-001). Renin antibody (#826 RKR—Rat Renal Renin) was kindly gifted by Dr. Tadashi Inagami, Vanderbilt University School of Medicine, Nashville, Tenn.

The inventors have investigated the role of hyperangiotensinemia in SCD pathophysiology and the associated organ damage, such as SCD-associated nephropathy (SN). SN is a leading cause of mortality in adults with SCD and has been presumed to occur from sickling-associated vaso-occlusions; hence the underlying molecular mechanisms are unexplored and no targeted therapies exist.

SCD-associated renal pathologies begin in childhood with loss of urine concentrating ability (UCA), a relatively unique feature of SN. By adulthood, 30-50% patients develop glomerulopathy, characterized as focal segmental glomerulosclerosis, which results in progressively increasing albuminuria and renal insufficiency (FIG. 2).

The inventors found that hyperangiotensinemia was essential for preserving the UCA in SCD mice, but also led to increased TGFβ1 production over time, resulting in glomerulopathy. Genetic deficiency of angiotensin receptor-1 (AT1R) signaling in the kidneys, or its pharmacological blockade in SCD mice completely abrogated the glomerulopathy, but further impaired UCA. UCA was even worse with angiotensin-converting-enzyme inhibition compared to AT1R-blockade because both AT1R and AT2R maintained UCA in SCD mice. Indeed, the impairment in UCA from AT1R-blockade was reversed by concomitant stimulation of AT2R (see FIGS. 2 and 3).

The inventors found a functional AT1R in circulating erythrocytes, where it activated Rac, to generate high amounts of reactive oxygen species (ROS), specifically in sickle erythrocytes. AT1R-induced sickle erythrocyte ROS, in turn, increased angiotensin-II, via a positive feedback loop between ROS, Angiotensin-II and AT1R. AT1R played no role in ROS-generation in SCD platelets (FIG. 3-4).

Genetic knock-out of AT1R in SCD mice, but not in normal mice, remarkably decreased erythrocyte ROS, oxidized angiotensinogen and abrogating SCD glomerulopathy, although severe hyposthenuria compromised survival. Erythroid-specific AT1R deficiency in SCD mice, however, reduced RBC ROS, reversed SN in entirety, including the severe hyposthenuria induced by a global AT1R deficiency (FIG. 4).

Overall, they demonstrate that hyperangiotensinemia in SCD is mediated and perpetuated by AT1R signaling in sickle erythrocytes. While necessary for UCA, it eventually results in SCD glomerulopathy. Both erythrocyte ROS and SN can be ameliorated by an erythroid-specific AT1R deficiency, or pharmacologically, with concomitant blockade of AT1R and stimulation of AT2R signaling.

Angiotensin II is generated by cleavage of its precursor molecule angiotensinogen (ANG) by the action of renin. ANG levels in plasma were found to be comparable in Berk-SS and control mice. Renin levels in plasma or in glomeruli of Berk-SS mice were also comparable to normal controls in young SCD mice (<24 weeks); they were elevated in older SCD mice, compared to age matched controls, suggesting that hyperreninemia was not the primary cause of hyperangiotensinemia (FIG. 5).

The inventors investigated the temporal consequence of hyperangiotensinemia on SCD renal pathophysiology in two mouse models of SCD that closely mimic human SCD phenotype, Berk-SS mice and Knock-in SCD mice (Knock in-SS, where the corresponding human globin genes have been knocked in place of the mouse globin genes). In contrast, their normal counterparts (Berk-AA and Knock-in AA mice) resembled WT C57BI/6 mice. Like humans with SCD, SCD mice developed loss of UCA. Albuminuria developed by young adulthood and peaked thereafter due to the high mortality in severely affected mice. Young Berk-SS mice showed glomerular hyperfiltration which rapidly declined with age, as is seen in human patients with SCD. Berk-SS kidneys showed RBC congestion, hemosiderosis, mononuclear infiltration, and areas of cystic necrosis as previously described in mice and mesangial proliferation and focal segmented glomerulosclerosis (FSGS) as reported in human patients. Indeed, renal pathology seen in a SCD patient who had macroalbuminuria and underwent a renal biopsy was very similar to that seen in SCD mice (FIG. 6).

In order to determine the role of the angiotensin receptors in SN, the inventors generated hematopoietic chimeric animals through bone marrow (BM) transplantation. First, they transplanted BM from Berk-SS mice into WT [057BI/6 (CD45.2+) or congenic CD45.1+BI/6] mice; mice that developed >95% sickle chimerism were followed for 6-9 months. Berk-SS/WT chimeras also developed hyperangiotensinemia followed by SN. Like in native (untransplanted) sickle mice, losartan or captopril ameliorated glomerular disease, but worsened UCA of hematopoietic SCD chimeras. These studies also demonstrated that hyperangiotensinemia and resultant SN was instigated by sickle hematopoiesis and was therefore transplantable (FIG. 7).

Remarkably high ROS were seen in erythrocytes and platelets in both SCD mouse models compared to normal erythrocytes and platelets. The same phenomenon was recapitulated in human subjects with SCD, compared to their unaffected siblings. However, there was no significant difference in ROS production by circulating leukocytes in either Berk-SS mice, Knock-in SS mice or in human subjects with SCD as compared to their corresponding normal controls, suggesting that phagocytes were not major effectors of hematopoietic ROS in SCD, and the majority of oxidative stress was mediated via erythrocytes, followed by platelets (FIG. 7).

The inventors found that blockade of AT₁R signalling by either losartan or captopril significantly lowered erythrocyte ROS in SCD mice, while platelet ROS was unaffected (FIG. 4); and the same phenomenon of significantly lowered erythrocyte ROS and unchanged platelet ROS was seen with losartan/captopril treated Berk-SS/WT hematopoietic chimeras. The data suggest that platelet ROS elevation is not associated to AT₁R signalling and may be secondary to hemodynamic changes induced by Ang-II (FIG. 8).

Besides captopril, losartan treatment also reduced plasma and urine Ang-II levels (FIGS. 4 and 8); and both captopril and losartan specifically lowered sickle erythrocyte ROS production with equal efficacy. These data suggested that a significant amount of sickle erythrocyte oxidative stress may derive from AT₁R signalling. If that were the case, Ang-II mediated AT₁R signalling in sickle erythrocytes may mediate a positive feedback loop to increase ROS production and perpetuate hyperangiotensinemia.

The inventors confirmed the pharmacological data in genetic knockouts by evaluating erythrocyte ROS production in AT₁R^{-/-} mice. Surprisingly, there was no significant difference in erythrocyte ROS or superoxide production in WT (non-SCD) AT₁R^{-/-} mice compared to WT littermates (AT₁R^{+/+} mice). To determine whether AT₁R signalling was specific to sickle erythroid cells, they generated SCD mice genetically deficient in AT₁R by interbreeding. Only occasional Berk-SS/AT₁R^{-/-} mice were successfully obtained, but all of them died by 5-6 weeks of age, likely due to severe loss of UCA. Knock-in SS/AT₁R^{-/-} mice showed a significant reduction in ROS production and superoxide generation in circulating erythrocytes (FIGS. 4 and 8).

These changes were erythroid cell-specific because ROS production did not significantly change in AT₁R deficient sickle leukocytes. Indeed, AT₁R signalling contributed to nearly one-half to one-third of the total ROS in sickle erythrocytes. The same phenomenon was seen in the two Berk-SS/AT₁R^{-/-} mice obtained. No change was observed in ROS on platelets in SCD mice with or without AT₁R, confirming that in SCD, platelet ROS is generated by mechanisms other than AT₁R signalling (FIGS. 4 and 8).

1-16. (canceled)

17. A method of treating sickle cell disease, comprising administering a therapeutically effective amount of an AT2 receptor agonist, or a pharmaceutically acceptable salt, or solvate thereof, to a subject suffering from sickle cell disease.

18. The method of claim 17, wherein the AT2 receptor agonist, or a pharmaceutically acceptable salt, or solvate thereof, is administered in combination with:
(i) an AT1 receptor antagonist, or a pharmaceutically acceptable salt, or solvate thereof; and/or
(ii) an inhibitor of angiotensin converting enzyme (ACE), or a pharmaceutically acceptable salt, or solvate thereof.

19. The method of claim 17, wherein the AT2 receptor agonist is a selective agonist of the AT2 receptor.

20. The method of claim 17, wherein the AT2 receptor agonist is N-butyloxycarbonyl-3-(4-imidazol-1-ylmethylphenyl)-5 so-butylthiophene-2-sulfonamide, or a pharmaceutically acceptable salt, or solvate thereof.

21. The method of claim 17, wherein the AT2 receptor agonist is provided in the form of N-butyloxycarbonyl-3-(4-imidazol-1-ylmethylphenyl)-5-iso-butylthiophene-2-sulfonamide free base or the HCl salt thereof.

22. The method of claim 17, wherein the AT2 receptor agonist is a compound having an anti-nephropathic effect with a reduction in end organ damage to the kidneys.

23. The method of claim 17, wherein the AT2 receptor agonist is a compound that reduces sickle cell disease-associated nephropathy.

24. The method of claim 17, wherein the subject a human subject.

25. The method of claim 17, wherein the treatment of sickle cell disease results in an improvement in the urine concentrating ability in a subject.

26. A method of improving urine concentrating ability in a subject having sickle cell anemia, comprising administering to a subject in need thereof, a therapeutically effective amount of an AT2 receptor agonist, or a pharmaceutically acceptable salt, or solvate thereof, thereby improving urine concentrating ability in the subject having sickle cell anemia as compared to a subject having sickle cell anemia and not administered the therapeutically effective amount of an AT2 receptor agonist, or a pharmaceutically acceptable salt, or solvate thereof.

27. The method of claim 26, wherein the AT2 receptor agonist, or a pharmaceutically acceptable salt, or solvate thereof, is administered in combination with:
(i) an AT1 receptor antagonist, or a pharmaceutically acceptable salt, or solvate thereof; and/or
(ii) an inhibitor of angiotensin converting enzyme (ACE), or a pharmaceutically acceptable salt, or solvate thereof.

28. The method of claim 26, wherein the AT2 receptor agonist is a selective agonist of the AT2 receptor.

29. The method of claim 26, wherein the AT2 receptor agonist is N-butyloxycarbonyl-3-(4-imidazol-1-ylmethylphenyl)-5-iso-butylthiophene-2-sulfonamide, or a pharmaceutically acceptable salt, or solvate thereof.

30. The method of claim 26, wherein the AT2 receptor agonist is provided in the form of N-butyloxycarbonyl-3-(4-imidazol-1-ylmethylphenyl)-5-iso-butylthiophene-2-sulfonamide free base or the HCl salt thereof.

31. The method of claim 26, wherein the AT2 receptor agonist is a compound having an anti-nephropathic effect with a reduction in end organ damage to the kidneys.

32. The method of claim 26, wherein the AT2 receptor agonist is a compound that reduces sickle cell disease-associated nephropathy.

33. The method of claim 26, wherein the subject a human subject.

34. A kit for treating sickle cell disease or for improving urine concentrating ability in a subject having sickle cell disease, comprising as separate components:
(a) a pharmaceutical formulation comprising an angiotensin II receptor agonist, or a pharmaceutically acceptable salt, or solvate thereof, in admixture with a pharmaceutically-acceptable adjuvant, diluent and/or carrier; and
(b) a pharmaceutical formulation comprising an AT1 receptor antagonist, or a pharmaceutically acceptable salt, or solvate thereof, and/or an ACE inhibitor, or a pharmaceutically acceptable salt, or solvate thereof, in admixture with a pharmaceutically-acceptable adjuvant, diluent and/or carrier, wherein components (a) and (b) are each provided in a form that is suitable for administration in conjunction with the other.
